(19) United States
(12) Patent Application Publication
(10) Pub. No.: US 2001/0034488 A1
(43) Pub. Date: Oct. 25, 2001

(54) Method and system of automated hemodynamical detection of arrhythmias

(76) Inventors: Shal Policker, Moshav Zur Moshe (IL); Itzhak Shemer, Haifa (IL); Yuval Mika, Zichron Yaakov (IL); Tamar Harel, Haifa (IL)

Correspondence Address: William H. Dippert, Cowan, Leibowitz & Latman, P.C., 1133 Avenue of the Americas, New York, NY, 10036-6799, US

(21) Appl. No.: 09/776,632

(22) Filed: Feb. 5, 2001

Related U.S. Application Data
(60) Provisional application No. 60/181,244, filed on Feb. 9, 2000.

(51) Int. Cl. A61B 5/0468

(52) U.S. Cl. 600/515

(57) ABSTRACT

A method for automated detection of arrhythmias in a patient is disclosed, said method comprising:

obtaining continuous pressure data from a cardiac chamber of said patient;

segmenting said pressure data to correspond to beat segments;

extracting features from each of said beat segments with respect to a predetermined number of earlier beat segments, said features relating to at least some of the criteria selected from: beat-to-beat change in the duration of said beat segments, beat-to-beat change in the peak to-peak amplitude of said beat segments, beat-to-boat change in heart rate of said patient and change in value and instance in time of max dP/dt, where P is pressure and t is time; and

classifying each of said beat segments as ectopic or normal, wherein ectopic beat is determined using a set of predetermined rules aimed at detecting abnormality in features of each of said beat segments with reference to predetermined thresholds.

### FIELD OF THE INVENTION

The present invention relates to the detection of cardiac arrhythmia. More particularly it relates to an automated method and apparatus for the detection of cardiac arrhythmia by analyzing a hemodynamic waveform. The present application corresponds to U.S. Provisional Patent Application Ser. No. 60/181,244, filed 02/09/2000.

### BACKGROUND OF THE INVENTION

The normal heart rhythm is meticulously regulated by the sinoatrial node (S A node), which is located at the right atrium of the heart. The SA node generates electrical signals which propagate along predetermined pathways along the cardiac muscle and cause the heart to contract, thus pumping blood throughout the body.

Cardiac arrhythmia is an abnormal condition of the heart where irregularity occurs in the normal heart rhythm. Arrhythmia may be caused by the failure of the cardiac electrical system to generate proper impulses, or it may appear when abnormal foci within the heart interfere with the normal impulse sequence initiating short-term or longer-term electrical signals.

Arrhythmias may be benign, symptomatic, life threatening or even fatal. Their consequences depend not only on their manifestation but on the presence of important abnormal structural conditions of the heart.

Most people may experience single episodes of a premature beat (PB). Atrial episodes of a single PB are usually harmless and the heart easily resumes its normal pace to overcome the problem. If the premature beat originates in the atrial zones of the heart it is called supraventricular premature beat (SPB), which actually refers to all premature beat events not originating from the ventricle, and sometimes premature supraventricular contraction (PSC), and if it originates in the ventricle it is called VPR3 (ventricle premature beat) or premature ventricular contraction (PVC).

Although both types of arrhythmias may bring about serious cardiac condition it is thought that ventricular arrhythmias are potentially more dangerous as the hemodynamic activity in the ventricles is much greater than the hemodynamic activity in the atria.

It is customary to define and differentiate between several kinds of arrhythmias, given here is their order of gravity (see: “What is an arrhythmia”, St. Luke's-Roosevelt Hospital Center, on the world wide web, http://www.arrhythmia.org/general/index.html):

Atrial tachycardia is an electric focus (or electric circuit) originating in the atrial chambers of the heart. It interferes with the normal electrical behaviour of the atria sending rapid electrical signals across he atrial chambers.

Atrial flutter is caused by rapid generation of electrical foci but with a fairly regular rhythm maintained due to reentry of the electrical impulse within the atria.

Atrial fibrillation is a chaotic rapid cardiac rhythm originating from multiple sites within the atria. Electrical foci exist simultaneously within the atria, in a dynamically changing pattern. As a result, rapid impulses attempt to cross the AV node into the ventricles. The AV node allows only some of the se impulses 4 cross. acting as a natural filter in an attempt to prevent excessive excitation of the heart. Nevertheless the ventricles will experience more rapid stimuli and irregular rhythm will occur.

AV nodal reentrant tachycardia is the most common form of paroxysmal supraventricular tachycardia or PSVT. Patients afflicted with this arrhythmia do not usually have other structural problems with their heart. The arrhythmia originates in the tissues near the AV node, the electrical structure that transmits impulses between the upper and lower chambers of the heart. Susceptible individuals will have two pathways that can channel impulses to and from the AV node. Under the right conditions, usually following a premature beat, these pathways can form an electrical circuit. An impulse will revolve around this circuit and each revolution will lead to impulse propagation to the ventricles, and thus a rapid heart beat.

Ventricular tachycardia is an arrhythmia that originates in the pumping chambers, or the ventricles. It is usually seen in patients who have damaged ventricular chambers, frequently in the aftermath of a heart attack or myocardial infarction. Scar tissue in the ventricles will alter many local electrical properties and set up conditions favorable to formation of a local electrical circuit. Under specific circumstances, the circuit can be activated leading to a rapid arrhythmia arising from a single spot within the pumping chambers. Because this is more rapid than the heart's natural electrical activity, it tales over the heart beat for the duration of the arrhythmia. Because it is so rapid, and is occurring in a damaged heart, and because the electrical sequence does not follow the normal pattern, the heart may not function properly or efficiently and low bloodpressure may result.

In its most extreme form ventricular tachycardia can lead to fatal consequences. This is a potentially dangerous arrhythmia that almost always requires therapy. In some patients, ventricular tachycardia may occur when there is no structural heart disease. This “idiopathic” form often arises from the right ventricle and less often from the left ventricle. These arrhythmias are less dangerous, but also often require therapy.

Conventionally arrhythmia is diagnosed by a medical professional upon obtaining and analyzing ECG or EGM results of the patient. It is noted that both measuring devices are electrical—ECG reading global electrical signals and EGM reading more localized electrical signals. Either way the final diagnosis is decided and given by the medical professional.

Devices detecting arrhythmias are also known. For example, various pacemaker devices are capable of detecting various types of bradyarrhythmia (also known as bradycardia) and provide artificial pacing therapy to one or more cardiac chambers. See, for example, U.S. Pat. No. 3,648,707 (Greatbatch), titled MULTIMODE CARDIAC PACES WITH P-WAVE AND R-WAVE SENSING MEANS, filed in 1969, and U.S. Pat. No. 5,161,529 (Stotts et al.), titled CARDIAC PACEMAKER WITH CAPTURE VERIFICATION, filed in 1988.

Other types of anti-arrhythmic devices such as cardiac defibrillators and defibrillator/cardioverter devices are known, which are designed to detect various different types of tachy-arrhythmia (also known as tachycardia) such as ventricular tachycardia (VT) and ventricular fibrillation (VF), and to provide one or more types of appropriate anti-tachycardia therapy to the heart such as anti-tachycardia pacing (ATP) therapy and shock defibrillation therapy, respectively. Such devices may use multi-tiered tachy-arrhythmia detection algorithms (also known as classification algorithms) for distinguishing between VT, VF and supraventricular tachycardia (SVT) arising from atrial fibrillation and for applying the proper type of therapy selected from ATP therapy, (low or medium energy shock therapy, and high energy defibrillating shock therapy. See, for example, U.S. Pat. No. 4,403,614 (Engle et al.), titled IMPLANTABLE CARDIOVERTER, filed in 1981.

All of the aforementioned methods and devices for detecting arrhythmias rely on the detection of the electrical activity of the myocardium, collecting readings from predetermined regions of the heart, and analyzing the electrical data to determine the occurrence of arrhythmia in the patient.

It is desirable to develop a new method of determining the occurrence of arrhythmias which does not rely on detecting electrical activity, but rather seeks to detect other detectable physical parameters, which imply the occurrence of an arrhythmic event, and therefore erable the diagnosis of arrhythmias.

The need for such new method stems from the fact that electrical measurement devices are prone to noise and background interference and it is desirable to find an independent method of determining arrhythmias that does no involve measuring the electrical activity of the myocardium.

Moreover, new such method for the determination of arrhythmias, can be employed in conjunction with other method or methods of determining arrhythmias based on electrical measurements, to provide cross verification and therefore facilitate a more precise diagnosis of arrhythmias, less prone to false results.

It is an object of the present Invention to provide a novel method and device for the detection of arrhythmias by measuring and analyzing pressure changes within the chambers of the heart.

Furthermore, it is another purpose of the present invention to provide such method for the detection of arrhythmias, based on collecting and analyzing physical parameters other than the electrical activity of the myocardium.

Moreover, it is another purpose of the present invention to provide such a method that measures the pressure within a cardiac chamber and processes the retrieved data to determine the occurrence of arrhythmias.

Measurements of the rate d change of the heart's pressure (dP/dt) are not new and were carried out, for example for the determination of a need to speed up pacemaker signal rate. See, for example, “FalsePositive Behaviour with the dP/dt sensing Pacemaker a rare complication of a physiological sensor”, by Crossley et al, PACE, Vol, 20, p. 2492., or “A Noninvasive Method of Measuring Max(dP/dt) of the Left Ventricle by Doppler Echocardiography”, by Senda et al., J. of Biomechanical Engineering, Vol. 114/15. Analysis algorithms for LVP are reported by B. R. Hieb et al, Left Ventricular Pressure Analysis: Design and Validation of a Computer Algorithm with an Investigation of Inter-Physician Variability, Computers and Biomedical Research 11, 229241 (1978) with no reference to arrhythmia detection.

### BRIEF DESCRIPTION OF THE INVENTION

There is thus provided a method for automated detection of arrhythmias in a patient, said method comprising:

obtaining a hemodynamic data signal corresponding to a continuous pressure data from a cardiac chamber of said patient;

segmenting said pressure data to correspond to beat segments;

extracting hemodynamic features from each of said beat segments with respect to a predetermined number of earlier beat segments, said hemodynamic features relating to the hemodynamic data signal; and

classifying each of said beat segments as ectopic or normal wherein ectopic beat is determined using a set of predetermined rules aimed at detecting abnormality in features of each of said beat segments with reference to predetermined thresholds.

Furthermore, in accordance with another preferred embodiment of the present invention, the hemodynamic features correspond to at least some of the criteria selected from: beat-to-beat change in the duration of said beat segments, beat-to-beat change in the peak-to-peak amplitude of said beat segments, beat-to-beat change in heart rate of said patient as measured in the time difference between two instances of max dP/dt of each two consecutive beats and change in value and instance in time of max dP/dt, where P is pressure and t is time

Furthermore, in accordance with another preferred embodiment of the present invention, said max dP/dt is calculated as the first derivative d sub-segments of the pressure beat segment, after smoothing of the beat.

Furthermore, in accordance with another preferred embodiment of the present invention, said smoothing is done using Savitzki-Golay filtering.

Furthermore, in accordance with another preferred embodiment of the present invention, a beat segment is classified as ectopic when a deviation of not less than 10% from norm hemodynamic values is detected.

Furthermore, in accordance with another preferred embodiment of the present invention, the obtaining of continuous pressure data from a cardiac chamber of said patient comprises positioning a pressure sensor within a cardiac chamber of the patient, and reading continuous pressure data by a reader electrically connected to the pressure sensor.

Furthermore, in accordance with another preferred embodiment of the present invention, said segmenting said pressure reading to correspond to beats segments comprises determining adaptive zero crossing.

Furthermore, in accordance with another preferred embodiment of the present invention, said segmenting said pressure reading to correspond to beats segments comprises:

estimating of the DC offset of said beats; and

subtracting the estimated DC offset from said beat to find two zero crossing points.

Furthermore, in accordance with another preferred embodiment of the present invention, said estimating of the DC offset of said beats comprises:

marking all local maximas within the segment and above a predetermined first threshold value;

choosing a point which is the lowest among all local maxima;

marking all local minimas within the segment and below a predetermined second threshold value;

choosing a point which is the highest among all local minima; and

calculating the DC offset as the average value between the lowest local maximum and highest local minimum.

Furthermore, in accordance with another preferred embodiment of the present invention, said first threshold value is in the range of 20-30 mmHg.

Furthermore, in accordance with another preferred embodiment of the present invention, said second threshold value is in the range of 30-40 mmHg.

Furthermore, in accordance with another preferred embodiment of the present invention, the method further comprises:

determining whether the time lapsed between each pair of adjacent zero crossing points is not less then 40 msec; and

excluding the pair of zero crossing point that occurred less then 40 msec from previous pair of zero crossing points.

Furthermore, in accordance with another preferred embodiment of the present invention, one hemodynaic feature relates to a norm value that corresponds to the end systolic pressure value defined as the highest value of the pressure data,

Furthermore, in accordance with another preferred embodiment of the present invention, the end systolic pressure is determined by:

finding the minimal absolute derivative in the range of the maximum pressure beat;

determining the maximum of the beat;

centering a window of about 50 msec centered about said maximum beat; and

calculating the smoothed absolute first derivative in the range of said window, and determining the end systolic pressure as the minimum value.

Furthermore, in accordance with another preferred embodiment of the present invention, said peak-to-peak amplitude is calculated by subtracting the lowest plateau pressure value of the beat from the end systolic pressure value.

Furthermore, in accordance with another preferred embodiment of the present invention, there is provided a system for the automated detection of arrhythmias in a patient, said system comprising:

a pressure sensor adapted to be positioned within a patient's cardiac chamber;

a processor communicating with said pressure sensor, said processor adapted to obtain continuous pressure data from said pressure sensor, segment said pressure data to correspond to beat segments, extract features from each of said beat segments with respect to a predetermined number of earlier beat segments, said features relating to at least some of the criteria selected from: beat-to-beat change in the duration of said beat segments, beat-to-beat change in the peak-to-peak amplitude of said beat segments, beat-to-beat change in heart rate of said patient and change in value and instance in time of max dP/dt, where P is pressure and t is time, and classify each of said beat segments as ectopic or normal, wherein ectopic beat is determined using a set of predetermined rules aimed at detecting abnormality in features of each of said beat segments with reference to predetermined thresholds.

### BRIEF DESCRIPTION OF THE FIGURES

In order to better understand the present invention, and appreciate its practical applications, the following Figures are provided and referenced hereafter. It should be noted that the Figures are given as examples only and in no way limit the scope of the invention as defined in the appending Claims. Like components are denoted by like reference numerals.

FIG. 1 is a charted illustration of a typical LVP signal, pinpointing some of its features.

FIG. 2 illustrates a general block diagram of the method for determination of arrhythmias, in accordance with a preferred embodiment of the present invention

FIG. 3 illustrates a system for the determination of arrhythmias, in accordance with a preferred embodiment of the present invention,

### DETAILED DESCRIPTION OF THE INVENTION AND FIGURES

The hemodynamic activity within the heart is usually correlated to the electrical activity of the myocardium Each contraction of the cardiac muscle, a result of a depolarization wave propagating through the myocardial tissue, brings about a rise in the internal pressure within the cardiac chamber experiencing the contraction, be it the one of the atria or ventricles.

It is therefore anticipated that for each heart beat, a rise of pressure within one of the hearts chambers that undergoes contraction will occur, that would be followed by a consequent drop of pressure as the chamber returns to its original relaxed condition (hereafter referred to as the pressure cycle).

A main postulate at the foundation of the present invention is the realization that it is possible to determine the occurrence of arrhythmias by monitoring the hemodynamic condition within the heart. More particularly it is suggested that a measurement of the pressure condition within a cardiac chamber be conducted over a lengthy period of time and determine the occurrence of arrhythmias by observing irregularities in the pressure cycle and analyzing them by computerized means employing a novel algorithm as described hereafter,

The implementation of the method and employment of the device of the present invention are intended both for chronic use in patients with known cardiac disorders, who need the implantation of a pacemaker or an ETC device, or for short term use, such as during an open chest cardiac surgery, or for postsurgery use, when the cardiac rhythm may be volatile.

The problem of automatic arrhythmia detection is well known and several solutions were introduced in the part (see, for example U.S. Pat. No. 4,403,614 (Engle et al.), titled IMPLANTABLE CARDIOVERTER, filed in 1981, and Nonpharmacological Therapy of Arrhythmias for the 21^{st} Century—the state of the art (Singer, Barold and Camm, Futura Pub., 1998, ch. 17, p. 386423). These methods generally rely on electrical signals as the input information, be it global (ECG) or local (intracardiac electrode leads). Recently carried out clinical tests show that electric signals measurement in some patients are prone to substantial disturbances and distortions, and render these measurements unsuitable for the purpose of automatic detection of arrhythmia. It is claimed that as much as 1653% of false positive diagnosed arrhythmia occur using known arrhythmia electrical detection methods, and that may lead to serious proarrhythmic consequences, with reported incidences ranging up to 8% of patients.

For this reason the method of detection of arrhythmia in accordance with the present invention was developed.

Another reason for the development of the system and method of the present invention is related to ETC, as disclosed in PCT/lL97/00012 (published as WO 97/25098), titled ELECTRICAL MUSCLE CONTROL (Ber-Haim et al.) incorporated herein by reference. The cardiac environment, undergoing ETC treatment is very noisy, and this reduces the reliability of ECG as a source for determining arrhythmias.

The proposed method and device may be also used as a verification means, in conjunction with other method of detection.

It is suggested to sense the pressure in any of the cardiac chambers, preferably from one of the ventricles and use the retrieved pressure data as the input data which is to be processed and/or analyzed.

For the purpose of bravity left ventriclar pressure (LVP) is considered. Note that the scope of the invention is not limited to LVP measurements only, as pressure measurements from the right ventricle or other cardiac chambers are also suited for arrhythmia detection according to the method and apparatus of the present invention LVP may be measured using implantable catheter having a pressure sensor at its distal end, or any other means for the measurement of pressure within cardiac chambers.

It was found that LVP is strongly related to the cardiac excitation state, rising during contraction of the myocardium and dropping when the cardiac muscle relaxed. Moreover when the cardiac contraction is normal in its strength, as expected in normal viable conditions the rise and consequent drop in LVP are significantly large showing a substantial LVP change, whereas when the contraction is weak, as in the cases of tachycardia or fibrillation, LVP changes are significantly smaller.

Left ventricular pressure (LVP) is used for the assessment of the mechanical hemodynamic performance of he heart, Parameters like LV end-diastolic pressure (LVEDP), LV peak systolic pressure (LVPSP), peak rate of LV pressure change during isovolumic contraction (+maxdP/dt) and relaxation (−dP/dt) are usually extracted from the measured LVP waveform. These parameters are used for clinical evaluation of left ventricular contractility in a variety of patients with different diseases (Braunwald E., Ross J. Jr, Sonnenblick E. H., “Methods for assessing contractility”, Mechanisms of Contraction of the Normal and Failing Heart, Edition 2, Little, Brown and Company, Boston 1976, pp. 130165), Some of these parameters to are commonly monitored in coronary care units (CCU) and intensive care units (ICU). Some pacemakers use the information gathered from the LVP signal for different applications. For instance, the +maxdP/dt parameter is used as a heart rate change indicator in a rate adaptive pacemaker. (Crossley G. H., Kiger L. A., Haisty W. K., Simmons T. W., Zmijewski M., Fitzgerald D. M., “False Positive Behavior with the dP/dt Sensing Pacemaker: A Rare Complication of a Physiological Sensor”, PACE, 20, pp. 2492-2495, 1997).

It is suggested to use the LVP signal for automatic detection of arrhythmias, an application which this signal has not been used for so far. The hemodynamic activity within the heart is strongly correlated with the electrical activity of the myocardium. Each contraction and relaxation of the cardiac muscle, which is a result of depolarization and repolarization waves transferred through the electrical conducting system of the heart, causes a rise in the internal pressure within the cardiac chambers (atria and/or ventricle), and a consequent drop of pressure as the chamber returns to its original relaxed condition. It is therefore anticipated that disturbances in the normal electrical conductance of the heart will be manifested as irregular changes in the LVP waveform. A decrease in contractility has already been reported by Steinbach et al (Steinbach K. K., Merl O.,Frohner K., Heif C., Numberg M., Kaltenbrunner W., Podczeck A., Vessely E., “Hemodynamics during ventricular tachyarrhythmias”, Am. Heart J., 127, pp. 1102-6, 1994). during ventricular tachyarrhythmias. Since contractility is measured by +maxdP/dt (Senda S., Sugawara M., Matsumoto Y., Kan T., Matsuo H., “A Noninvasive Method of Measuring Max(dP/dt) of the Left ventricle by Doppler Echocardiography”, J. Biochem. Eng., 114, pp. 15-19,1992), a change in LVP waveform is highly likely to be observed during tachyarrhythmias.

Automatic arrhythmia detection has been the subject of extensive work, due to the need of a fast response when life threatening events occur. Currently methods and devices used for detecting arrhythmias are based on ECG or electrogram analysis. ECG based algorithms are found in cardiac monitors in coronary care units (CCU's) and intensive care units (ICU's). Electrogram based algorithms are found in pacemaker devices that are capable of detecting various types of bradycardia (Scotts et al. U.S. Pat. 5,161,529, “Cardiac Pacemaker with Capture Verification”, filed in 1988), and antitachycardia and cardioverter defibrillator devices that use rate and morphology variations in the intracardiac electrograms for discrimination between sinus rhythm and arrhythmic beats,(Dicario L. A., Throne R. D., Jenkins J. M, “A TimeDomain Analysis of Intracardiac Electrograms for Arrhythmia Detection”, PACE, 14, pp. 329-336, 1991). Unfortunately, electrical signals measurements are prone to substantial disturbances and distortions in some patients, hence rendering these measurement, in some cases be unsuitable for the purpose of automatic detection of arrhythmia. Hernandez et al. (Hernandez A. I., Carrault G., Mora F., Thoraval L., Passariello G., Schleich J. M., “Multisensor Fusion for Atrial and Ventricular Activity Detection in Coronary Care Monitoring”, IEEE Biomed. Eng., 46, pp. 1186-1190, 1999) claim that most systems in CCU's still produce undesirable alarms and are prone to confuse artifacts with rhythms. Therefore, these systems may become ineffective in some cases, and make human observation and monitoring mandatory In implantable defibrillators 16-53% of false positive diagnosed arrhythmias occur using known current detection methods. This may lead to serious proarrhythmic consequences, with reported incidences ranging up to 8% of patients. (Duffin E. G., “Future Implantable Defibrillator Technologies”, pp. 437-455, In Nonpharmacological Therapy of Arrhythmias for the 21^{st} Century, The State of the Art, by Singer I., Barold S. S. and Camm J., Futura Publishing Company, Inc., N.Y., 1998).

It is therefore suggested to find an independent method for determining arrhythmias that does not involve measuring the electrical activity of the myocardium. Moreover, it is suggested to use this method in conjunction with other existing methods for arrhythmia detection. The incorporation of results from two independent methods, one method based on the analysis of electrical activity and the other based on the analysis of mechanical activity, will provide cross verification and therefore facilitate a more precise diagnosis for arrhythmias, less prone to false to results.

A main aspect of the method for the determination of arrhythmia of the present invention is the assumption that an automatic segmentation process should be used for dividing the pressure waveform into segments where each segment represents a single heart beats. The relevant parameters considered in the determination of arrhythmia in each beat segment are.

1. substantial increase of the beat-to-beat rate of a patient relative to his recently measured beat rate (in a window of between 5 to 50 last beats), which normally varies in the range of 50-180 beats pet minute;

2. widening of the pulse curve shape following the last maximum dP/dt point, and

3. substantial decrease in variation in peak-to-peak amplitude.

The basic rate for the LVP signal, which represents the heart beat, correspond to the location of the maximum rate of pressure change (maxdP/dt) in each cycle. There are three shape parameters for each cycle that correspond to the time periods between a max dP/dt point and the down slope crossing of the 25%, 50% and 75% of the peak-to-peak threshold. These parameters are useful in detecting extra-systoles that are close to the original systole, and thus appear on the down slope of the pressure pulse curve, failing to be segmented as separate pulses, Peak-to-peak values are calculated using an algorithm that does not take into account the global extreme values of the beat cycle, but detects periods of low first-difference around these values.

The algorithm is based on dividing the LVP signal into segments, where each segment contains a single beat cycle. Each single beat cycle is characterized as a normal or ectopic beat according to the following criteria: Change in the width of the beat above a certain threshold; Relative change in the peat-to-peak (PTP) amplitude; and Relative change in heart rate.

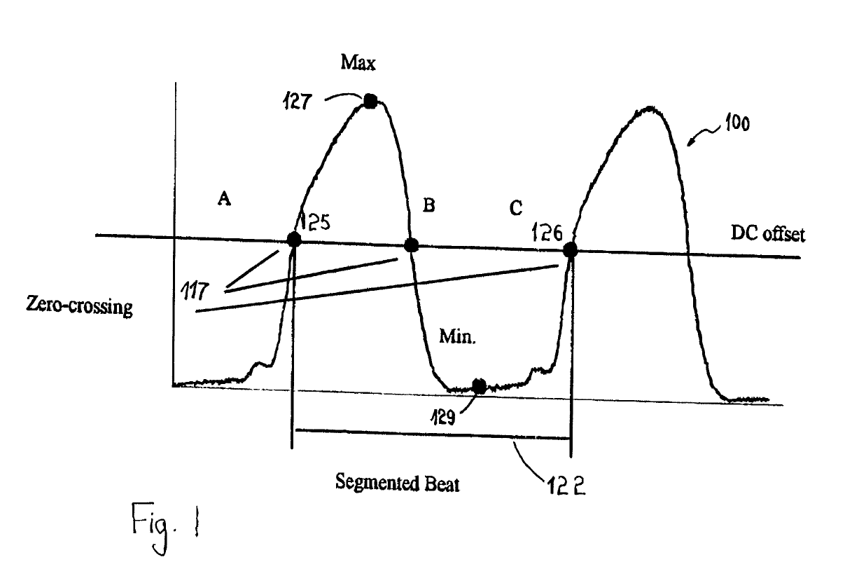

A main aspect of the method of the present invention is to divide the whole LVP signal 100, collected from a patient by placing a pressure sensor in one of Ibe patient's cardial chambers, preferably in one of the ventricles, into single beat cycles, or segments (segmented beat cycle 122). The estimated beginning and end of each beat cycle are near the maximum 125 and minimum 126 slopes of the analyzed beat, as depicted by FIG. 1. The maximum point is denoted by 127 and the minimum point is denoted by 129.

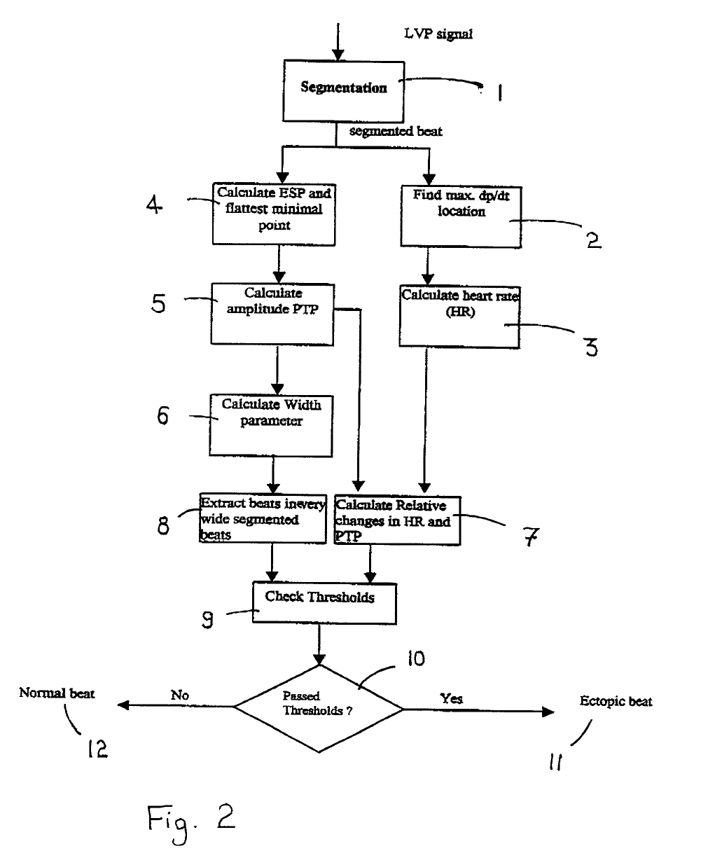

A general block diagram of the method for determination of arrhythmias, in accordance with a preferred embodiment of the present invention is illustrated in FIG. 2.

The beat segmentation 1 in the present invention is based on adaptive zero crossing, and is divided into two main steps:

1. Estimation of the DC offset of the current beat

2. Subtraction of the DC offset from the current beat to find the two zero crossing points.

Estimation of the DC offset of the current segment is intended to maximize performance of a zero crossing method in an adaptive manner The DC offset is calculated by first marking all local maximas within the segment and above a predetermined threshold (for example 30-40 mmHg). Then we choose a point which is the lowest among all local maxima. This point serves as an estimation of the maximum value of the beat (marked as Max. 127 in FIG. 1). The same analysis is done for the local minimas which are below another predetermined threshold (for example(20-30 mmHg) to estimate the highest value of the local minima (marked as Min. 129 in FIG. 1). Hence, the DC offset is calculated as the average between the lowest local maximum and highest local minimum values of the analyzed beat.

Subtraction of the DC offset from the analyzed beat is conducted to find the zero crossing points 117. The calculated DC offset is subtracted from the analyzed beat, and the zero crossing points are searched and marked. Finally, each adjacent pairs of zero crossing points are checked, to see whether there is a local minimal (or maxima) between them, and whether there are at least separated by 40 msec. If not, the first zero crossing point of the pair is excluded from the list of zero crossing points, and the search continues,

Hence, the output of this stage of the algorithm is a list of zero crossing points, which divide the whole LVP signal into beat cycles, as depicted in FIG. 1.

Calculation of End Systolic Pressure (ESP) 4 value is done as follows. The End Systolic Pressure (ESP) value of the LVP beat is defined as the highest plateau of the LVP beat. The calculation of the ESP is based on finding the minimal absolute derivative in the range of the maximum LVP beat. In order to find the ESP point, the maximum of the LVP beat is found. A window length of 50 msec is centered about the location of this maximum. The smoothed absolute first derivative of the signal in the range of this window is estimated. The minimum value of this estimation is defined as the maximal plateau−the ESP value.

The peak-to-peak (PTP) amplitude value 5 of the LVP signal is then calculated using the following equation:

PTP=ESP−lowest plateau of LVP beat

The lowest plateau of the LVP beat is calculated similarly to the way the ESP is calculated. There are two main differences between the calculation of ESP and the calculation of the lowest plateau: instead of calculating the maximum value of the LVP beat, the minimum value is calculated. Heart rate is calculated as the time difference between adjacent locations of the maximum upward slope of the LVP signal (maxdP/dt). Estimating maxdP/dt 2 for each beat cycle is based on data smoothing, and calculating the first derivative of subsegments of the smoothed LVP beat. maxdP/dt is defined as the maximum value of the calculated derivatives. An important feature in this algorithm is the smoothing of the data. This is done by using the Savitzky-Golay (SG) filter. The Savitzky-Golay filter is an FIR smoothing filter that is optimal for smoothing out noisy signal whose frequency span (without noise) is large (Press W. H., Teukolsky S. A., Vetterling W. T., Flannery B. P., “Numerical; Recipes in C”,pp. 650-655, Cambridge University Press, 1995). It replaces each data value fᵢ by a polynorn fit (minimum least squares) of order m of nearby neighboring samples. Its main advantage is that it is most effective in preserving high derivative components, while still smoothing out significant portion of the noise. Hence, using this filter enables data smoothing, while still preserving the first derivatives of the signal. The derivatives are calculated over subsegments windows, preferably of about 15 msec duration, Current methods used today for the calculation of maxdP/dt use a running 5-point weighted slope for the digital differentiation of the LVP signal (Kass D. A., Chen C. H., Curry C., Talbot M., Berger R., Fetics B., Nevo E., “Improved Left Ventricular Mechanics From Acute VDD Pacing in Patients With Dilated Cardiomyopethy and Ventricular Conduction Delay”, Circulation, 99, pp. 1567-1573, 1999) or apply a logistic model to the LVP signal, and calculate its derivative.(Matsubara H., Takaki M., Yasuhara S., Arakt J., Suga H., “Logistic Time Constant of Isovolumic Relaxation Pressure-Time Curve in the Canine Left Ventricle”, Circulation, 92, pp. 2318-2326, 1995). The proposed algorithm, which is based on the SG smoothing filter is believed to provide more accurate results, since it is less biased by noise.

The next step is extracting width parameters 6 (the duration of the beat) for each LVP beat. The width of the LVP beat can be calculated, for example, as the time difference between every two adjacent lowest plateau points or the time difference between the max dP/dt point and the next plateau point.

The relative change in each of the two parameters (HR or PTP) 7 is then calculated, it being the difference between the parameter value of the current beat and the median parameter value of the last 7 previous beats. The relative changes calculated in percentage.

The next step is to decide whether a beat is normal 12 or ectopic 11 beat. Four criterions are observed and considered in order to decide whether a beat is ectopic or normal;

1) Time lapsed between the current beat and its previous and following beat

if the time lapsed between the analyzed beat and the previous beat is shorter than a threshold (LowLimitHR), and the time lapsed between the analyzed beat and the following beat is greater than a threshold (UpLimitHR), the beat s classified as an ectopic beat. The thresholds are determined as 10% smaller (LowLimitHR) or higher (UpLimitHR) than the mean heart rate.

2) Relative change in HR

A relative change in heart rate of the current analyzed beat that is higher than 8% classifies the beat as an ectopic beat.

3) Relative change in PTP

A relative change in PTP amplitude of the current beat that is smaller than a threshold (example: 3·σ_{median PTP}) classifies the beat as an ectopic beat.

4) Width of the current beat

A beat that is wider than a predetermined threshold (for example: 1.5·median(beatswidths)) is classified as a wide beat. A wide beat may be a result of consecutive beats, where the first one is a normal beat and the remaining beats are ectopic beats. This phenomenon is also known as couplets or triplets. The segmentation algorithm may fail to distinguish two or three separate beats when this phenomena occurs, hence one wide beat is observed. An algorithm based on the calculation of the first and second smoothed derivatives is applied to find the instances of time when the ectopic beats occur within the detected wide beat.

To conclude: if one of the above criteria exists—the analyzed beat is classified as an ectopic beat.

The patients participating in a study carried out by the inventors were HF patients undergoing an EP study in a cardiac electrophysiology (EP) laboratory. Pacing and sense electrodes were placed in the high right atrium (RA) and the right ventricle (RV) apex. A CARDIMA® electrode was inserted through the subclavian vein, and enabled the recording of left ventricle local sense (LVLS). A double sensor catheter 5F Millar, #SPC-571 Millar instruments, Tex., USA, was used for the measurement of left ventricular pressure (LVP). ECG signal (lead II) was also measured. All measured signals were sampled with 12 bits A/D, 1kHz sampling rate. During the study the patients were paced in DDI mode. The pacemaker rate to was set to be 10% higher than the normal sinus rhythm of the patient, and the AV delay was set to be 15%-20% less than the paced AV delay. Hence, the patients were typically atrial and ventricular paced.

An offline analysis of the LVP signals was conducted to detect ectopic beats and validate the method of the present invention. Twelve saved files containing is readings (˜9500 beats) of different HF patients were selected randomly and served as the database for the evaluation of the method of the present invention. The ECG and LVLS traces were analyzed off hard by a specialist to mark the arrhythmic beats. Ectopic beats were defined by their location relative to the pacing artifacts sensed in the LVLS and by their aberrant PQRS pattern. Pacemaker fault—pacemaker mediated aberrant beats (i.e. escape beats after pacemaker inhibited beats, early single WI beat following noise in pacemaker sense channels), were not defined as ectopic beats.

Only the LVP signal was analyzed by the automatic algorithm, without any knowledge of the pacing times, or ECG or LVLS signals changes. The results of the automatic algorithm analysis were compared to the specialists offline analysis.

The algorithm of the present invention was tested and had produced 64 false positive (FP) beats out of 9102 normal beats (0.7%) and 38 false negative (FN) out of 268 ectopic beats (14.2%) for a total detection failure of 102 beats (1%)- The algorithm detected ectopic beats of atrial and ventricular origin. Most of the failures of the algorithm in detecting ectopic beats occurred when a supraventricular beat occurred, and as a result the pacemaker did not pace the atrium. On the other hand, early beats that occurred as a result of a pacemaker fault, were detected as arrhythmic beats. These beats were not classified as ectopic beats by the offline analysis, hence contributed to the false positive rate of the algorithm. A pre knowledge of pacing times can prevent the detection of these beats as ectopic, and reduce the FP rate. In some cases the algorithm was found to be more sensitive than the ECG signal, as depicted in FIG. 2. chart A illustrates an ECG chart obtained from a patient and chart B illustrates a synchronized LVP signal obtained from the same patient.

No morphological change is seen in the ECG trace shown in chart A. The change in local heart rate is barely seen, whereas the change in the LVP amplitude in chart B is clearly visible and obvious. LVP amplitude in chart B is measured in mmHg.

The algorithm presented herein is a recommended mode of operation according to the present invention, but is not the only possible way to exercise the method for the detection of arrhythmias of the present invention. As mentioned earlier the method of the present invention is based on automatic analysis of LVP signals. The parameters that were found to describe best the important changes in the LVP signals were heart rate, amplitude (PTP), and width of the beats.

The thresholds for the parameters were found according to the distribution of the parameters in each file, hence adapting the values of toe thresholds to each patient separately.

One of the major drawbacks of the algorithm of the present invention is that it analyzes data that is obtained by cardiac catheterization, an invasive method. Hence, it is applicable only to devices and situations Where catheterization is a procedure that is done anyhow. The idea of integrating complementary data from hemodynamic signals with the usual electrical signals, to enhance the reliability of current methods of cardiac rhythm monitoring has been lately suggested by Hernandez et al. Although being of heterogeneous nature, both the ECG and hemodynamic signals, such as LVP, have information mutually correlated. This is due to the interrelation of the mechanical and the electrical functions of the heart Hernandez et al. claim that since data of different physiological sources is acquired anyway in CCU/ICU, the fusion of the different types of data is obvious, and can definitely improve existing methods for rhythm analysis. This idea can be further extended to other application such as pacemakers control and cardioverter defibrillators. A better arrhythmic detection can prevent false detection that may lead to serious proarrhythmic consequences. Moreover, the joint evaluation of the electrical stimuli as well as the mechanical response can lead to an optimization of the energy of the stimuli given by the cardioverter defibrillator. An idea that must be investigated is whether the use of combined data only improves the detection of arrhythmic beats or also enables to forecast the occurrence of these arrhythmias. It is suggested, for example to incorporate the algorithm of the method for determination of arrhythmias of the present invention in an ETC (Excitable Tissue Control) apparatus, as disclosed in PCT/IL97/00012 (published as WO 97/25098), titled ELECTRICAL MUSCLE CONTROL (BemHaim et al.) incorporated herein by reference. This may serve to improve the accuracy of the data input of the apparatus, thus improve its performance and increase its efficiency

Although the algorithm discussed herein made use of LVP signal as input, the method of the present invention is not limited in its input data to LVP signal only. Other cardiac pressure readings may be considered suitable for analysis, i.e. RVP (Right Ventricle Pressure), LAP (Left Atrial Pressure) or RAP (Right Atrial Pressure) or aortic blood pressure

Another advantage of the method of the present invention is the ability to conduct an on-line or offline analysis of pressure data collected from the patient (in-vivo results or cardiac history).

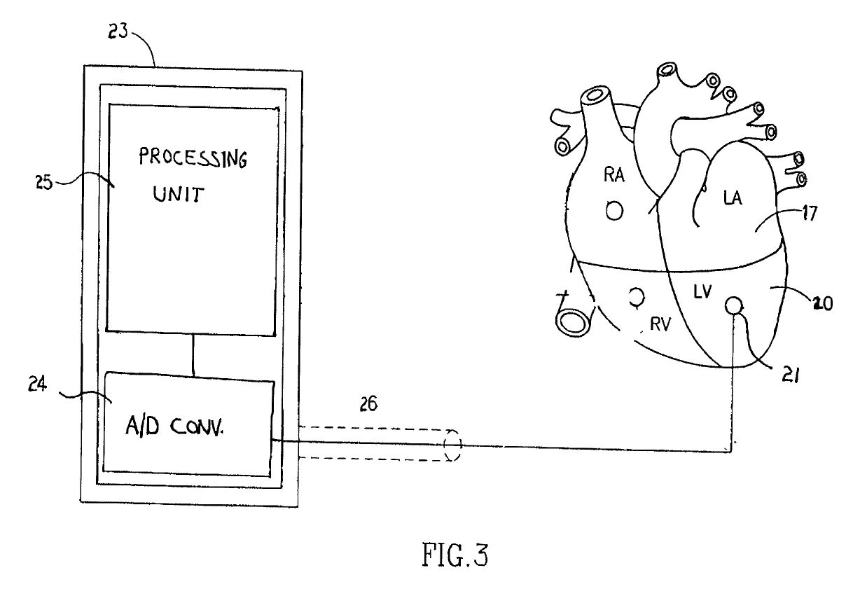

FIG. 3 illustrates a system for the determination of arrhythmias, in accordance with a preferred embodiment of the present invention. A pressure sensor 21 is positioned, using a catheter 26, and placed in a patient's heat 17, preferably inside the left ventricle 20. The pressure sensor 21 is electrically connected to a reader 23 comprising an analog-to-digital converter 24, which converts the analog signal from the sensor to digital data, The digital data is processed by a processing unit 25, adapted to process the data using an algorithm for the automated detection of arrhythmias, in accordance with the present invention, as discussed above

It should be clear that the description of the embodiments and attached Figures set forth in this specification serves only for a better understanding of the invention, without limiting its scope as covered by the following Claims.

It should also be clear that a person skilled in the art, after reading the present specification could make adjustments or amendments to the attached Figures and above described embodiments that would still be covered by the following Claims.

1. A method for automated detection of arrhythmias in a patient said method comprising:
obtaining a hemodynamic data signal corresponding to a continuous pressure data from a cardiac chamber of said patient;
segmenting said pressure data to correspond to beat segments;
extracting hemodynamic features from each of said beat segments with respect to a predetermined number of earlier beat segments, said hemodynamic features relating to the hemodynamic data signal, and
classifying each of said beat segments as ectopic or normal wherein ectopic beat is determined using a set of predetermined rules aimed at detecting abnormality in features of each of said beat segments with reference to predetermined thresholds.

2. The method as claimed in claim 1, wherein the hemodynamic features correspond to at least some of the criteria selected from. beat-to-beat change in the duration of said beat segments, beat-to-beat change in the peak-to-peak amplitude of said beat segments, beat-to-beat change in heart rate of said patient as measured in the time difference between two instances of max dP/dt of each two consecutive beats and change in value and instance in time of max dP/dt, where P is pressure and t is time.

3. The method as claimed in claim 2, wherein said max dP/dt is calculated as the first derivative of sub-segments of the pressure beat segment, after smoothing of the beat.

4. The method as claimed in claim 3, wherein said smoothing is done using Savitzki-Golay filtering.

5. The method as claimed in claim 1, wherein a beat segment is classified as ectopic when a deviation of not less than 10% from norm hemodynamic values is detected.

6. The method as claimed in claim 1 wherein the obtaining of continuous pressure data from a cardiac chamber of said patient comprises positioning a pressure sensor within a cardiac chamber of the patient, and reading continuous pressure data by a reader electrically connected to the pressure sensor.

7. The Method as claimed in claim 1, wherein said segmenting said pressure reading to correspond to beats segments comprises determining adaptive zero crossing.

8. The method as claimed in claim 1, wherein said segmenting said pressure reading to correspond to beats segments comprises,
estimating of the DC offset of said beats; and
subtracting the estimated DC offset from said beat to find two zero crossing points.

9. The method as claimed in claim 8, wherein said estimating of the DC offset of said beats comprises:
marking all local maximas within the segment and above a predetermined first threshold value;
choosing a point which is the lowest among all local maxima;
marking all local minimas within the segment and below a predetermined second threshold value;
choosing a point which is the highest among all local minima; and
calculating the DC offset as the average value between the lowest local maximum and highest local minimum.

10. The method as claimed in claim 9, wherein said first threshold value is in the range of 20-30 mmHg.

11. The method as claimed in claim 9, wherein said second threshold value is in the range of 30-40 mmHg.

12. The method as claimed in claim 8 further comprising:
determining whether the time lapsed between each pair of adjacent zero crossing points is not less then 40 msec; and
excluding the pair of zero crossing point that occurred less then 40 msec from previous pair of zero crossing points.

13. The method as claimed in claim 1, wherein one hemodynaic feature relates to a norm value that corresponds to the end systolic pressure value defined as the highest value of the pressure data.

14. The method as claimed in claim 13, wherein the end systolic pressure is determined by:
finding the minimal absolute derivative in the range of the maximum pressure beat;
determining the maximum of the beat;
centering a window of about 50 msec centered about said maximum beat; and
calculating the smoothed absolute first derivative in the range of said window, and determining the end systolic pressure as the minimum value.

15. The method as claimed in claim 14, wherein said peak-to-peak amplitude is calculated by subtracting the lowest plateau pressure value of the beat from the end systolic pressure value.

16. A system for the automated detection of arrhythmias in a patient, said system comprising:
a pressure sensor adapted to be positioned within a patient's cardiac chamber;
a processor communicating with said pressure sensor, said processor to adapted to obtain continuous pressure data from said pressure sensor, segment said pressure data to correspond to beat segments, extract features from each of said beat segments with respect to a predetermined number of earlier beat segments, said features relating to at least some of the criteria selected from: beat-to-beat change in the duration of said beat segments, beat-to-beat change in the peak-to-peak amplitude of said beat segments, beat-to-beat change in heart rate of said patient and change in value and instance in time of max dP/dt, where P is pressure and t is time, and classify each of said beat segments as ectopic or normal, wherein ectopic beat is determined using a set of predetermined rules aimed at detecting abnormality in features of each of said beat segments with reference to predetermined thresholds.
